(19) United States
(12) Patent Application Publication
(10) Pub. No.: US 2025/0387533 A1
(43) Pub. Date: Dec. 25, 2025

(54) TISSUE CLEANING SOLUTIONS AND RELATED METHODS

(71) Applicant: Tides Medical LLC, Lafayette, LA (US)

(72) Inventors: Mohammadali Safavieh, North Andover, MA (US); Mora Carolyn Melican, Weston, MA (US)

(73) Assignee: Tides Medical LLC, Lafayette, LA (US)

(21) Appl. No.: 18/752,662

(22) Filed: Jun. 24, 2024

(51) Int. Cl. A61L 27/36

(52) U.S. Cl. CPC A61L 27/3604; A61L 27/3687; A61L 27/3691; A61L 2430/34; A61L 2430/40

(57) ABSTRACT

This disclosure generally relates to methods of preparing a graft for wound healing. In some embodiments, the methods comprise obtaining a donor placenta and isolating tissue comprising amnion and/or chorion from the donor placenta. In other embodiments, the methods comprise washing the isolated tissue with a cleansing solution. Other embodiments, still, are directed toward applying a current to the isolated tissue and/or cleansing solution to produce the graft for wound healing.

### FIELD

The present invention generally relates to tissue cleaning solutions and related methods, such as tissue cleaning solutions for preparing grafts comprising amnion and/or chorion.

### BACKGROUND

Allografts derived from placental tissue, such as amnion grafts, chorion grafts, and amnion/chorion combination grafts, are commonly used as wound dressings, for example, to treat non-healing chronic wounds (e.g., wounds that fail to proceed through the normal phases of wound healing in an orderly and timely manner). One of the major challenges associated with using placental tissue as wound dressings is the need to properly wash the tissue prior to use. Washing is important to reduce the risk of infection and to ensure that the tissue is safe for use in a subject in need. However, cleansing reagents are often harsh and can damage the protein structure and/or physical integrity of certain placental tissues, especially, membrane-derived grafts such as amnion and chlorin grafts. Accordingly, improved solutions and methods are needed.

### SUMMARY

The subject matter of the present disclosure involves, in some cases, interrelated products, alternative solutions to a particular problem, and/or a plurality of different uses of one or more systems and/or articles.

Aspects of the disclosure generally relate to methods of preparing a graft for wound healing. In some embodiments, the methods relate to obtaining a donor placenta and isolating tissue comprising amnion and/or chorion from the donor placenta. In some embodiments, the methods relate to washing the isolated donor tissue with a cleansing solution. In some embodiments, the methods relate to applying a current to the isolated donor tissue and/or cleansing solution, thereby forming the graft.

In other aspects, the methods relate to administering a graft to a subject for prevention or treatment of a particular condition (e.g., non-healing chronic wounds). It is to be understood that in each such aspect of the disclosure, the disclosure specifically includes, also, the graft for use in the treatment or prevention of that particular condition, as well as use of the graft for the manufacture of a medicament for the treatment or prevention of that particular condition.

In another aspect, the present disclosure encompasses methods of making one or more of the embodiments described herein, for example, a graft. In still another aspect, the present disclosure encompasses methods of using one or more of the embodiments described herein, for example, a graft.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A-1C shows photographs of an amnion membrane isolated from a donor placenta before electrophoretic washing (FIG. 1A), 30 minutes after electrophoretic washing (FIG. 1B), and 120 minutes after electrophoretic washing (FIG. 1C), according to one set of embodiments. For example, as shown in FIGS. 1A-1C, electrophoretic washing increases the transparency of the amnion membrane, which is an indication that at least some of the cellular component of the amnion membrane has been removed. The photographs also show that the amnion membrane remains intact during the 120 minute wash.

FIG. 2A-2C shows photographs of a chorion membrane isolated from a donor placenta before electrophoretic washing (FIG. 2A), 30 minutes after electrophoretic washing (FIG. 2B), and 120 minutes after electrophoretic washing (FIG. 2C), according to one set of embodiments. For example, as shown in FIGS. 2A-2C, electrophoretic washing increases the transparency of the chorion membrane, which is an indication that at least some of the cellular component of the chorion membrane has been removed. The photographs also show that the chorion membrane remains intact during the 120 minute wash.

FIG. 3A-3D shows photographs comparing the effects of passive washing versus electrophoretic washing of a chorion membrane isolated from a donor placenta, according to one set of embodiments. Prior to the start of passive washing the chorion membrane was intact and clearly contained cellular components (as indicated by the discoloration of the membrane, FIG. 3A). Passive washing for 270 minutes increased the transparency of the chorion membrane but caused the chorion membrane to separate (e.g., tear), thus decreasing the structural integrity of the membrane itself (FIG. 3B). Similarly, prior to the start of electrophoretic washing, the chorion membrane was intact and contained cellular components (FIG. 3C). Electrophoretic washing for 270 minutes significantly increased the transparency of the chorion membrane while maintaining the structural integrity of the membrane itself (e.g., the membrane was not torn, FIG. 3D).

### DETAILED DESCRIPTION

This disclosure generally relates to methods of preparing a graft for wound healing. In some embodiments, the methods comprise obtaining a donor placenta and isolating tissue comprising amnion and/or chorion from the donor placenta. In other embodiments, the methods comprise washing the isolated donor tissue with a cleansing solution. Other embodiments, still, are directed toward applying a current to the isolated donor tissue and/or cleansing solution to produce the graft for wound healing.

Allografts derived from placental tissue, such as amnion grafts, chorion grafts, and amnion/chorion combination grafts, are commonly used as wound dressings, for example, to treat non-healing chronic wounds. Two of the major challenges associated with using donor placental tissue is the risk of rejection by the recipient and/or infection of the recipient from bacteria or viruses (e.g., HIV) present in the donor tissue. To mitigate this risk, placental tissues are generally extensively washed using a combination of detergents, enzymatic cleaners, and antimicrobial agents to remove said antigens from the placental tissue. Additionally, in some cases, the placental tissue may be further sterilized using electron beam (e-beam) or gamma (Y) sterilization, prior to use. However, these cleansing reagents and sterilization techniques are harsh and can damage the native protein architecture of certain placental tissues, especially, membrane-derived grafts such as amnion and chlorin grafts.

Accordingly, one aspect of the present disclosure is directed to the discovery that electrophoretic washing of an isolated placental tissue reduces and/or eliminates the presence of antigens while preserving the native protein architecture of the donated placental tissue (e.g., amnion graft, chorion graft, and/or amnion/chorion combination grafts). Without wishing to be bound by any particular theory, it is generally believed that antigens in the placental tissue are derived from the cellular component of the tissue and that clearing the cellular component of the placental tissue, using electrophoretic washing, reduces and/or eliminates the presence of said antigens within the placental tissue.

Electrophoretic washing is an art recognized technique commonly used to render complex biological tissues optically transparent for subsequent use in molecular imaging applications. It has been discovered and appreciated by the inventors of the present disclosure, however, that electrophoretic may be used to remove antigens from allografts. During electrophoretic washing, in some embodiments, the biological tissue is placed between a positive and negative electrode within an ionic detergent solution comprising ionic lipid micelles. Application of a current (e.g., via the negative electrode to the positive electrode) may, in some cases, cause the ionic lipid micelles to accelerate toward the oppositely charged electrode (e.g., cationic lipid micelles are accelerated toward a negative electrode and anionic lipid micelles are accelerated toward a positive electrode) causing the ionic lipid micelles to pass through the biological tissue before reaching the oppositely charged electrode. Without wishing to be bound by theory, as the ionic lipid micelles pass through the tissue, they interact with other lipid and lipid-like cellular components and incorporate them into their micellular structure. In this way, the ionic lipid micelles extract lipid and lipid-like cellular components from the cells within the biological tissue. In some embodiments, such extraction includes the lipid bilayer of the cell membrane, which is generally responsible for maintaining the cell integrity by separating the interior of the cell from the outside environment. The inventors discovered and appreciated that tuning of the washing procedure (e.g., number of wash cycles, temperature, current, etc.,) allows removal of most, if not all, of the cellular components of the tissue, including the cytoplasmic components without affecting the native structure of the tissue itself.

As such, some embodiments of the present disclosure relate to methods for preparing a graft for wound healing, for example, using electrophoretic washing. In some embodiments, the methods relate to obtaining a donor placenta and isolating tissue comprising amnion and/or chorion from the donor placenta. The methods, according to some embodiments, further relate to washing the isolated tissue with a cleansing solution (e.g., an ionic solution comprising ionic lipid micelles) and applying a current to the isolated tissue and/or cleansing solution, thereby yielding a washed graft.

Methods for obtaining a donor tissue are known to those skilled in the art, and include, for example harvesting of tissues from living and/or deceased donors. The donor tissue may be obtained from any suitable donor species. For example, in some embodiments the donor tissue is obtained from a human donor. In some cases, the human donor is deceased, however, it is noted that the human donor does not have to be deceased for donor tissue to be obtained (e.g., a placenta may be obtained after childbirth). In other words, in some embodiments, donor tissue is obtained from a living human donor. The living human donor, in some cases, may be obtained from oneself (e.g., a subject generally in need of said donor tissue), or alternatively, it may be obtained from a subject different than oneself, for example, via a human donor (e.g., a placental donation).

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a monkey, a gorilla, a whale, a rat, a hamster, a bird, a fish, or a guinea pig. Generally, the invention is directed toward use with humans. In some embodiments, a subject may demonstrate health benefits, e.g., upon administration of a graft (e.g., comprising donor tissue treated in accordance with the embodiments described herein) as described herein.

In other embodiments, the donor tissue is obtained from a non-human donor. Again, donor tissue may be obtained from either living or deceased non-human donors. Any suitable non-human donor known to the skilled artisan may be used to obtain a donor tissue as described herein. For example, in some embodiments the non-human donor is porcine or bovine, although other non-human mammals may also be suitable.

In some embodiments, the isolated donor tissue is obtained from placental tissue. In some embodiments, the isolated donor tissue obtained from placental tissue is amnion. In some embodiments, the isolated donor tissue obtained from placental tissue is chorion. In some embodiments, the isolated donor tissue obtained from placental tissue comprises a combination of amnion and chorion. In some embodiments, the isolated donor tissue comprising amnion and/or chorion comprises one or more layers. In some embodiments the one or more layers are the same (e.g., amnion/amnion or chorion/chorion). In some embodiments, the one or more layers or different (e.g., amnion/chorion or chorion/amnion). In some embodiments, the donor tissue obtained from placental tissue is umbilical cord tissue.

The methods, according to some embodiments, further relate to exposing the donor tissue (e.g., placental tissue such as amnion and/or chorion) to a fixative. In some embodiments, the donor tissue is exposed to the fixative prior to washing the isolated donor tissue with a cleansing solution. Any suitable fixative known to one of skill in the art may be used as the fixative in the methods disclosed herein. For example, in some embodiments, the fixative is ethanol, methanol, formalin, formaldehyde, paraformaldehyde, glutaraldehyde, osmium tetroxide, glyoxal, picric acid, mercuric chloride, acetone, acetic acid, potassium dichromate, Bouin's fixative, acrolein, and genipin, among others. Any suitable concentration (by weight) of fixative may be used. For example, in some embodiments, the concentration of the fixative is greater than or equal to 1 wt %, greater than or equal to 5 wt %, greater than or equal to 10 wt %, greater than or equal to 15 wt %, greater than or equal to 20 wt %, greater than or equal to 25 wt %, greater than or equal to 30 wt %, greater than or equal to 35 wt %, greater than or equal to 40 wt %, greater than or equal to 45 wt %, or greater than or equal to 50 wt %. In some embodiments, the concentration of the fixative is less than or equal to 50 wt %, less than or equal to 45 wt %, less than or equal to 40 wt %, less than or equal to 35 wt %, less than or equal to 30 wt %, less than or equal to 25 wt %, less than or equal to 20 wt %, less than or equal to 15 wt %, less than or equal to 10 wt %, less than or equal to 5 wt %, or less than or equal to 1 wt %. Combinations of the above recited ranges are also possible. For example, in some embodiments, the concentration of the fixative is greater than or equal to 1 wt % and less than or equal to 50 wt %.

The skilled artisan will appreciate, however, that the isolated donor tissue does not need to be fixed (e.g., the isolated donor tissue is not exposed to a fixative; the isolated donor tissue is exposed to a fixative but in amount unsuitable for fixing the isolated donor tissue such as a very dilute solution comprising the fixative). In some embodiments, the methods disclosed herein do not comprise exposing the isolated donor tissue to a fixative.

In some embodiments, the isolated donor tissue may be preserved, for example, after isolation from a donor placenta. Any suitable preservation method known by the skilled artisan may be used to preserve the isolated donor tissues disclosed herein. Exemplary embodiments include, but are not limited to, cryopreservation, lyophilization (also referred to as freeze-drying), air-drying, or a combination thereof. One of ordinary skill in the art will understand and appreciate that some preservation methods may further comprise exposing the isolated donor tissue to one or more compounds intended to preserve the native structure and composition of the donor tissue (e.g., amnion and/or chorion). For example, during cryopreservation, the isolated donor tissue may be embedded in a cryoprotectant (e.g., dimethyl sulfoxide, DMSO, glycerol, sugar solutions, etc.) prior to freezing.

In some embodiments, the isolated donor tissue (e.g., amnion and/or chorion) is shaped into a graft that corresponds to the shape of a wound. Thus, in some embodiments the isolated donor tissue (e.g., amnion and/or chorion) is formed into a geometric shape. As used herein, the term “geometric shape” is given its ordinary meaning in the art and generally refers to an area closed by a boundary which is created by combining a specific number of curves, points, and lines. The isolated donor tissue (e.g., amnion and/or chorion) may be formed into any geometric shape known in the art, such as for example, regular geometric shapes and/or irregular geometric shapes (e.g., irregular circles). As used herein, the term “irregular geometric shape” generally refers to a any regular geometric shape with at least one distorted angle, point, and/or line. Exemplary geometric shapes include for example, squares, circles, rectangles, triangles, polygons, parallelogram, irregular squares, irregular circles, irregular rectangles, irregular triangles, irregular polygons, and/or irregular parallelograms. Other shapes are also contemplated in other embodiments.

The methods, in some embodiments, further relate to washing the isolated donor tissue with a cleansing solution. In some embodiments, the cleansing solution comprises a surfactant. As used herein the term “surfactant” is given its ordinary meaning in the art. In some embodiments, the surfactant comprises an amphipathic compound. Any suitable surfactant known to one of skill in the art may be incorporated into the cleansing solutions disclosed herein. For example, in some embodiments, the surfactant is an anionic surfactant. In some embodiments, the surfactant is a cationic surfactant. In some embodiments, the surfactant is a lipid. The lipid may further be a cationic lipid or an anionic lipid, according to some embodiments. In some embodiments, the anionic lipid is sodium dodecyl sulfate (SDS). In some embodiments, the anionic lipid is deoxycholate. Any suitable concentration of surfactant (e.g., SDS or deoxycholate) may be used in the cleansing solutions disclosed herein. For example, in some embodiments, the concentration of surfactant is greater than or equal to 0.001 mg/mL, greater than or equal to 0.005 mg/mL, greater than or equal to 0.01 mg/mL, greater than or equal to 0.05 mg/mL, greater than or equal to 0.1 mg/mL, greater than or equal to 0.5 mg/mL, 1 mg/mL, greater than or equal to 5 mg/mL, greater than or equal to 10 mg/mL, greater than or equal to 15 mg/mL, greater than or equal to 20 mg/mL, greater than or equal to 25 mg/mL, greater than or equal to 30 mg/mL, greater than or equal to 35 mg/mL, greater than or equal to 40 mg/mL, greater than or equal to 45 mg/mL, greater than or equal to 50 mg/mL, greater than or equal to 55 mg/mL, greater than or equal to 60 mg/mL, greater than or equal to 70 mg/mL, greater than or equal to 80 mg/mL, greater than or equal to 90 mg/mL, or greater than or equal to 100 mg/mL (e.g., per milliliter of water or saline). in some embodiments, the concentration of surfactant is less than or equal to 100 mg/mL, less than or equal to 90 mg/mL, less than or equal to 80 mg/mL, less than or equal to 70 mg/mL, less than or equal to 60 mg/mL, less than or equal to 55 mg/mL, less than or equal to 50 mg/mL, less than or equal to 45 mg/mL, less than or equal to 40 mg/mL, less than or equal to 35 mg/mL, less than or equal to 30 mg/mL, less than or equal to 25 mg/mL, less than or equal to 20 mg/mL, less than or equal to 15 mg/mL, less than or equal to 10 mg/mL, less than or equal to 5 mg/mL, less than or equal to 1 mg/mL, less than or equal to 0.5 mg/mL, less than or equal to 0.1 mg/mL, less than or equal to 0.05 mg/mL, less than or equal to 0.01 mg/mL, less than or equal to 0.05 mg/mL, or less than or equal to 0.01 mg/mL (e.g., per milliliter of water or saline). Combinations of the above recited ranges are also possible. For example, in some embodiments the concentration of surfactant is greater than or equal to 0.001 mg/mL and less than or equal to 100 mg/mL.

In an exemplary set of embodiments, the concentration of surfactant is between 0.0055 g/ml and 0.06 g/mL. In an exemplary set of embodiments, the concentration of surfactant is greater than or equal to 0.0055 g/mL, greater than or equal to 0.056 g/mL, greater than or equal 0.057 g/mL, greater than or equal to g/mL, greater than or equal to 0.058 g/mL, greater than or equal to 0.059 g/mL, or greater than or equal to 0.06 g/mL. In an exemplary set of embodiments, the concentration of surfactant is less than or equal to 0.06 g/mL, less than or equal to 0.059 g/mL, less than or equal to 0.058 g/mL, less than or equal to 0.057 g/mL, less than or equal to 0.056 g/mL, or less than or equal to 0.055 g/mL. Combinations of the above recited ranges are also possible. For example, in some embodiments the concentration of surfactant is greater than or equal to 0.055 g/mL and less than or equal to 100 g/mL.

In some embodiments, a cleansing solution comprising a surfactant (e.g., SDS or deoxycholate) comprises a micelle (e.g., comprising the surfactant and/or another surfactant). Without wishing to be bound by theory, suitable surfactants for forming a micelle generally comprise a hydrophilic domain and a hydrophobic domain (e.g., which thereby renders them amphipathic). When placed in aqueous solutions above a certain concentration (e.g., above a critical micellar concentration, CMC), surfactants may orient themselves such that their hydrophilic domains associate with the hydrophilic solvent and their hydrophobic domains are isolated from said hydrophilic solvent. The resulting supramolecular assemblies may be spherical in shape, or alternatively, they may take the form of an ellipsoid, a cylinder, or a bilayer structure. The CMC may be the same or different for each surfactant used and can readily be determined in the literature or using art recognized techniques by those of ordinary skill in the art (e.g., via light scattering techniques such as dynamic light scattering). For example, the CMC is between about 2-4 mg/mL and between about 1-2 mg/mL for SDS and deoxycholate, respectively.

Further, it is to be understood that micelles composed of ionic surfactants are also ionic. Thus, in some embodiments, the cleansing solution comprises ionic micelles. In some embodiments, the ionic micelle is an anionic micelle. In some embodiments, the ionic micelle is a cationic micelle.

The cleansing solutions disclosed herein may further comprises one or more other components useful for preparing grafts from isolated donor tissues, as disclosed herein. For example, in some embodiments, the cleansing solutions comprise a detergent. Any suitable detergent known to the skilled artisan may be used in the cleansing solutions disclosed herein. For example, in some embodiments, the detergent comprises Triton X-100, Triton X-200, CHAPS ((3-((3-cholamidopropyl)dimethylammonio)-1-propanesulfonate)), and TBP (tributyl phosphate) Other detergents are also possible.

In some embodiments, the cleansing solution comprises an enzymatic cleaner. Any suitable enzymatic cleaner known to the skilled artisan may be used in the cleansing solutions disclosed herein. Exemplary embodiments include, but are not limited to, Ethylenediaminetetraacetic acid (EDTA), ethylene glycol-bis(β-aminoethyl ether)-N,N,N′,N′-tetraacetic acid (EGTA), trypsin, pepsin, as well as all known, and yet to be discovered, endonucleases and exonucleases.

In some embodiments, the cleansing solution comprises a therapeutic agent. In some embodiments, the therapeutic agent is an antimicrobial agent. Any suitable antimicrobial agent known to the skilled artisan may be used in the cleansing solutions disclosed herein. In some embodiments, the antimicrobial compound comprises a penicillin. Non-limiting examples include penicillin V, penicillin G, amoxicillin, amoxicillin/clavulonate, ampicillin, nafcillin, oxacillin, dicloxacillin, piperacillin, pipercillin/tazobactam, and the like. In some embodiments, the antimicrobial compound comprises a macrolide. Examples include, but are not limited to, azithromycin, clarithromycin, fidaxomicin, erythromycin, telithromycin, and the like. In some embodiments, the antimicrobial compound comprises a cephalosporin. Examples include, but are not limited to, cefacetril, cefradin, cefroxadin, cefaloglycin, cefaclor, cefalexin, cefadroxil, cefatrizin, cefazedon, cefapirin, ceftezol, cefazolin, cefazaflur, cefalotin, cefaloridin, cefalonium, and the like. In some embodiments, the antimicrobial compound comprises a fluoroquinolone. Examples include balofloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, and the like. In some embodiments, the antimicrobial compound comprises a beta-lactam. Examples include penams, carbapenams, clavams, penems, carbapenems, cephems, carbacephems, oxacephems, monobactams, and the like. Combinations of the above recited compounds are also possible (e.g., the antimicrobial may comprise a penicillin and a beta-lactam or a fluoroquinolone and a cephalosporin, etc.).

In some embodiments, the therapeutic agent is an antifungal agent. Any suitable antifungal agent known to the skilled artisan may be used in the cleansing solution disclosed herein. In some embodiments the antifungal agent is a polyene. Exemplary embodiments include amphotericin B, candicidin, filipin, hamycin, natamycin, nystatin, and rimocidin. In some embodiments, the antifungal agent is an azole.

In some embodiments, the azole is an imidazole. Exemplary imidazoles include, but are not limited to, bifonazole, butoconazole, clotrimazole, econazole, fenticonazole, isoconazole, ketoconazole, luliconazole, miconazole, omoconazole, oxiconazole, sertaconazole, sulconazole, and ticonazole. In some embodiments, the azole is a triazole. Exemplary triazoles include, but are not limited to, albaconazole, efinaconazole, epoxiconazole, fluconazole, isavuconazole, itraconazole, posaconazole, propiconazole, ravuconazole, terconazole, and voriconazole. In some embodiments, the azole is a thiazole, such as, for example, abafungin.

In some embodiments, the antifungal agent is an allylamine. Exemplary allylamines include, but are not limited to, butenafine, naftifine, and terbinafine.

In some embodiments, the antifungal agent is an echinocandin. Exemplary enchinocandins include, but are not limited to, anidulafungin, caspofungin, and micafungin.

Additionally, according to some embodiments, the cleansing solution comprises a saline solution (e.g., normal saline, physiological saline, or isotonic saline). In some embodiments, the saline solution is present in the cleansing solution at a final concentration of 0.9% (w/v). Other concentrations are also possible. For example, in some embodiments, the concentration of saline in the cleansing solution is greater than or equal to 0.9% (w/v), greater than or equal to 3% (w/v), greater than or equal to 5% (w/v), greater than or equal to 7% (w/v), or greater than or equal to 23% (w/v). In some embodiments, the concentration of saline in the cleansing solution is less than or equal to 23% (w/v), less than or equal to 7% (w/v), less than or equal to 5% (w/v), less than or equal to 3% (w/v), or less than or equal to 0.9% (w/v). Combinations of the above recited ranges are also possible. For example, in some embodiments, the concentration of saline in the cleansing solution is greater than or equal to 0.9% (w/v) and less than or equal to 23% (w/v).

The cleansing solution may further comprise a colloid. Any suitable colloid known to the skilled artisan may be included in the cleansing solutions disclosed herein. Exemplary colloids include, but are not limited to starch, dextran, gelatin, albumin, and fresh frozen plasma. In some embodiments, the colloid is present in the cleansing solution at a concentration of greater than or equal to 0.1% (w/v), greater than or equal to 0.5% (w/v), greater than or equal to 1% (w/v), greater than or equal to 3% (w/v), greater than or equal to 5% (w/v), greater than or equal to 10% (w/v), greater than or equal to 15% (w/v), or greater than or equal to 20% (w/v). In some embodiments, the colloid is present in the cleansing solution at a concentration of less than or equal to 20% (w/v), less than or equal to 15% (w/v), less than or equal to 10% (w/v), less than or equal to 5% (w/v), less than or equal to 3% (w/v), less than or equal to 1% (w/v), less than or equal to 0.5% (w/v), or less than or equal to 0.1% (w/v). Combinations of the above recited ranges are also possible. For example, in some embodiments the colloid is present in the cleansing solution at a concentration of greater than or equal to 0.1% (w/v) and less than or equal to 20% (w/v).

In some embodiments, a cleansing solution is used to passively wash the isolated donor tissue (e.g., amnion graft or chorion graft). The phrase “passively wash” as used herein generally refers to the washing, of e.g., tissue, in the absence of an electric field. Thus, in some embodiments, the cleansing solution may be stirred during passive washing of the isolated donor tissue. Without wishing to be bound by any particular theory it is generally believed that stirring the cleansing solution improves removal of cellular debris (e.g., antigens) from the isolated donor tissue.

Additionally, or alternatively, an isolated donor tissue may be actively washed in a cleansing solution using electrophoretic washing. As described above, electrophoretic washing comprises application of an electric current to the cleansing solution comprising the isolated donor tissue. In some embodiments, applying the electric current comprises applying a uniform electric field. However, it is noted that, a uniform electric field is not required. Thus, in some embodiments, applying the electric current comprises applying a non-uniform electric field. In some embodiments, the applied current is greater than or equal to 0.2 A, greater than or equal to 0.4 A, greater than or equal to 0.6 A, greater than or equal to 0.8 A, greater than or equal to 0.9 A, greater than or equal to 1 A, greater than or equal to 1.5 A, greater than or equal to 2 A, greater than or equal to 3 A, greater than or equal to 4 A, or greater than or equal to 5 A. In some embodiments, the applied current is less than or equal to 5 A, less than or equal to 4 A, less than or equal to 3 A, less than or equal to 2 A, less than or equal to 1.5 A, less than or equal to 1 A, less than or equal to 0.9 A, less than or equal 0.8 A, less than or equal to 0.6 A, less than or equal to 0.4 A, or less than or equal to 0.2 A. In some embodiments, the applied current is between 1 and 1.5 A. Combinations of the above recited ranges are also possible. For example, in some embodiments, the applied current is greater than or equal to 0.2 A or less than or equal to 5 A.

As described in more detail above, during electrophoretic washing, an isolated donor tissue is placed between a positive and negative electrode within a cleansing solution comprising ionic lipid micelles. Application of a current (e.g., via the negative electrode to the positive electrode) causes the ionic lipid micelles to accelerate toward the oppositely charged electrode (e.g., cationic lipid micelles are accelerated toward a negative electrode and anionic lipid micelles are accelerated toward a positive electrode) causing the ionic lipid micelles to pass through the biological tissue before reaching the oppositely charged electrode. In some embodiments, the ionic lipid micelle is an anionic lipid micelle and application of current to the cleansing solution propels the anionic lipid micelle toward the positive electrode. In some embodiments, as the anionic lipid micelle passes through the isolated donor tissue, the anionic lipid micelle extracts lipids from a lipid-bilayer of one or more cells, or cellular components, within the isolated donor tissue (e.g., amnion and/or chorion grafts), thus removing the cellular component (and hence the “antigens”) of the isolated donor tissue.

A cleansing solution may be perfused (e.g. circulated) at any suitable rate known to the skilled artisan during washing of isolated donor tissues (e.g., any rate that does not damage or destroy the donor tissue). For example, in some embodiments, the cleansing solution is perfused at greater than or equal to 10 rpm, greater than or equal to 25 rpm, greater than or equal to 50 rpm, greater than or equal to 75 rpm, or greater than or equal to 100 rpm during perfusion of the donor tissue. In some embodiments the cleansing solution is perfused at less than or equal to 100 rpm, less than or equal to 75 rpm, less than or equal to 50 rpm, less than or equal to 25 rpm, or less than or equal to 10 rpm during passive washing of the donor tissue. Combinations of the above cited ranges are also possible. For example, in some embodiments the cleansing solution is perfused at greater than or equal to 10 rpm and less than or equal to 100 rpm.

A cleansing solution may be perfused for any suitable amount of time during washing of isolated donor tissues. For example, in some embodiments, the isolated donor tissue may be perfused for greater than or equal to 1 minute, greater than or equal to 5 minutes, greater than or equal to 10 minutes, greater than or equal to 15 minutes, greater than or equal to 20 minutes, greater than or equal to 30 minutes, greater than or equal to 40 minutes, greater than or equal to 50 minutes, or greater than or equal to 60 minutes. In some embodiments, the isolated donor tissue may be perfused for less than or equal to 60 minutes, less than or equal to 50 minutes, less than or equal to 40 minutes, less than or equal to 30 minutes, less than or equal to 20 minutes, less than or equal to 15 minutes, less than or equal to 10 minutes, less than or equal to 5 minutes, or less than or equal to 1 minute. Combinations of the above cited ranges are also possible according to some embodiments. For example, in some embodiments the isolated donor tissue may be perfused for greater than or equal to one minute and less than or equal to 60 minutes.

An isolated donor tissues may be washed with a cleansing solution any suitable number of times during passive and/or electrophoretic washing. For example, in some embodiments, an isolated donor tissue is washed greater than 1, greater than 2, greater than 3, greater than 4, greater than 5, greater than 6, greater than 7, greater than 8, greater than 9, or greater than 10 times. In some embodiments, the isolated donor tissue is washed less than 10, less than 9, less than 8, less than 7, less than 6, less than 5, less than 4, less than 3, less than 2, or less than 1 times. Combinations of the above cited ranges are also possible, according to some embodiments. For example, in some cases, the isolated donor tissue is washed greater than 1 time but less than 10 times.

Additionally, in some embodiments, a passive and/or electrophoretic washing step is performed at a temperature of between 4° C. and 40° C. For example, in some embodiments the passive and/or electrophoretic washing step (active washing step) is performed at greater than or equal to 4° C., greater than or equal to 10° C., greater than or equal to 15° C., greater than or equal to 20° C., greater than or equal to 25° C. greater than or equal to 27° C., greater than or equal to 29° C., greater than or equal to 31° C., greater than or equal to 33° C., greater than or equal to 35° C., greater than or equal to 37° C., or greater than or equal to 40° C. In some embodiments, the passive and/or electrophoretic washing step is preformed at less than or equal to 40° C., less than or equal to 37° C., less than or equal to 35° C., less than or equal to 33° C., less than or equal to 31° C., less than or equal to 29° C., less than or equal to 27° C., less than or equal to 25° C., less than or equal to 20° C., less than or equal to 15° C., less than or equal to 10° C., or less than or equal to 4° C. Combinations of the above recited ranges are also possible. For example, in some embodiments, the passive and/or electrophoretic washing step is performed at greater than or equal to 4° C. or less than or equal to 37° C.

### EXAMPLES

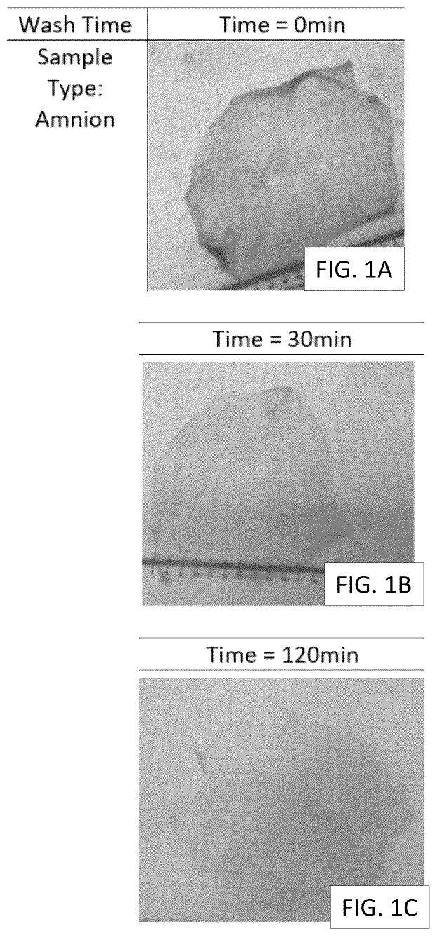

Example 1. Amnion membranes were thawed in a warm water bath, fixed in 4% w/v paraformaldehyde and stored at 4° C. overnight. Amnion membranes were then washed electrophoretically using a commercially available electrophoretic device and cleansing solution. The device settings were as follows: voltage: 70 volts, current: 1.5 amps, temperature: 37° C., and pump: 100 rpm. The tissues were washed for 30 minutes, photographs taken, and then placed back in the device for subsequent washings for up to 120 minutes. Inspection of FIGS. 1A-1C clearly shows that electrophoretic washing improved tissue clarity and maintained tissue integrity over the entire 120 minutes of wash time.

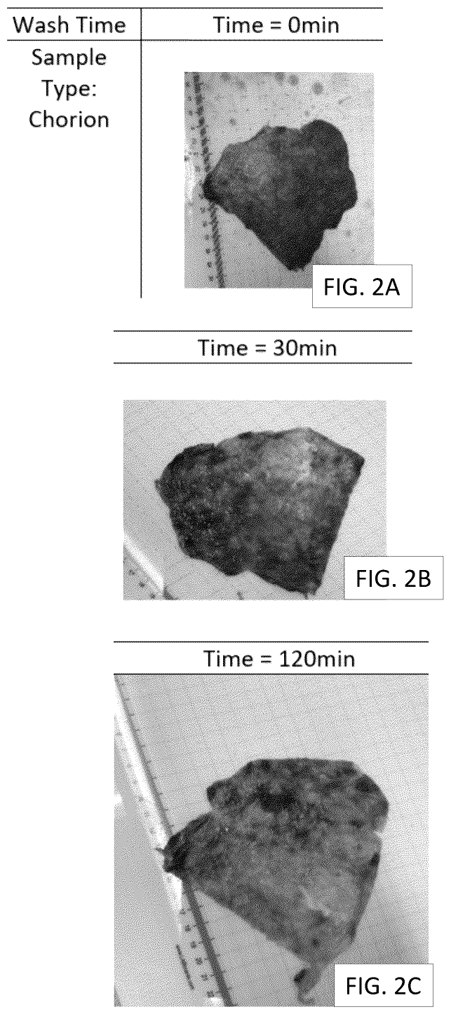

Example 2. Chorion membranes were thawed in a warm water bath, fixed in 4% w/v paraformaldehyde and stored at 4° C. overnight. Chorion membranes were then washed electrophoretically using a commercially available electrophoretic device and cleansing solution. The device settings were as follows: voltage: 70 volts, current: 1.5 amps, temperature: 37° C., and pump: 100 rpm. The tissues were washed for 30 minutes, photographs taken, and then placed back in the device for subsequent washings for up to 120 minutes. Inspection of FIGS. 2A-2C clearly show that electrophoretic washing improved tissue clarity and maintained tissue integrity over the entire 120 minutes of wash time.

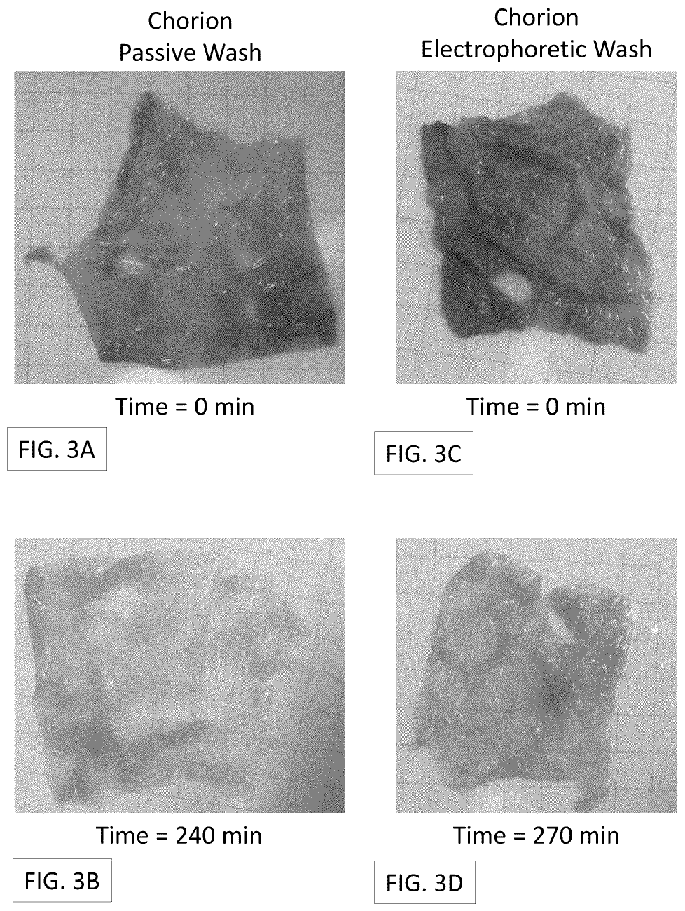

Example 3. Frozen chorion membrane was defrosted in a warm water bath. After thawing, the chorion membrane was cut into 2 sections, each having dimension of approximately 6×6 cm, and fixed in a solution of 10% w/v formaldehyde. The tissues were then washed either passively (e.g., in the presence of mixing only) or electrophoretically. A commercially available electrophoretic device was used for both passive and electrophoretic washing. For passive washing the device settings used were: temperature 22.9° C., pump speed: 100 rpm. For electrophoretic washing the device settings used were: voltage: 70 volts, current: 1.5 amps, temperature: 30° C., and pump: 100 rpm. The cleansing solution was also obtained from a commercially available vendor. The tissues were washed for 10 minutes, photographs taken, and then placed back in the device for subsequent washings. This process was repeated for between 240 minutes (for passive washing) and 270 minutes (for electrophoretic washing). Inspection of FIGS. 3A-3D clearly shows that passive washing improved tissue clarity, but introduced tears into the tissue. On the other hand, electrophoretic washing improved tissue clarity and maintained tissue integrity for up to 270 minutes of wash time.

### EQUIVALENTS AND SCOPE

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape-such as, round, square, gomboc, circular/circle, rectangular/rectangle, triangular/triangle, cylindrical/cylinder, elliptical/ellipse, (n) polygonal/(n) polygon, etc.; angular orientation-such as perpendicular, orthogonal, parallel, vertical, horizontal, collinear, etc.; contour and/or trajectory-such as, plane/planar, coplanar, hemispherical, semi-hemispherical, line/linear, hyperbolic, parabolic, flat, curved, straight, arcuate, sinusoidal, tangent/tangential, etc.; direction-such as, north, south, east, west, etc.; surface and/or bulk material properties and/or spatial/temporal resolution and/or distribution-such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed is:

1. A method of preparing a graft for wound healing, the method comprising:
obtaining a donor placenta and isolating tissue comprising amnion and/or chorion from the donor placenta;
washing the isolated donor tissue with a cleansing solution; and
applying a current to the isolated donor tissue and/or cleansing solution, thereby forming the graft.

2. The method of claim 1, further comprising exposing the isolated donor tissue to a fixative.

3. The method of claim 1, wherein the method of preparing the graft for wound healing does not comprise fixation of the isolated donor tissue.

4. The method of claim 1, wherein the isolated donor tissue comprising amnion and/or chorion comprises one or more layers, each of the one or more layers being the same or different.

5. The method of claim 1, wherein the isolated donor tissue comprising amnion and/or chorion is shaped into a graft that corresponds to the shape of the wound

6. The method of claim 1, wherein the cleansing solution comprises an anionic lipid.

7. The method of claim 6, wherein the anionic lipid is sodium dodecyl sulfate (SDS)

8. The method of claim 6, wherein the anionic lipid is deoxycholate

9. The method of claim 8, wherein the deoxycholate is present in the cleansing solution at a concentration of between 50 mg/mL and 60 mg/mL.

10. The method of claim 6, wherein the anionic lipid forms an anionic lipid micelle.

11. The method of claim 6, wherein the anionic lipid micelle extracts a lipid-bilayer from one or more cells, or cellular components, within the isolated donor tissue comprising amnion and/or chlorin.

12. The method of claim 1, wherein the cleansing solution comprising saline

13. The method of claim 1, wherein the step of applying the current propels the anionic lipid micelle towards the positive electrode.

14. The method of claim 1, wherein the step of applying the current comprises applying a uniform electric field.

15. The method of claim 1, wherein the applied current is between 1 and 1.5 amps.

16. The method of claim 1, wherein one or more steps of the method is conducted at a temperature range of between 4° C. and 40° C.
